(19) United States
(12) Patent Application Publication
(10) Pub. No.: US 2026/0363810 A1
(43) Pub. Date: Nov. 19, 2026

(54) SYSTEMS AND METHODS FOR CONTROL OF A SURGICAL SYSTEM

(71) Applicant: Intuitive Surgical Operations, Inc., Sunnyvale, CA (US)

(72) Inventors: Pedro REYNOSO-MORA, San Jose, CA (US); Paul G. GRIFFITHS, Santa Clara, CA (US)

(73) Assignee: Intuitive Surgical Operations, Inc., Sunnyvale, CA (US)

(21) Appl. No.: 19/148,101

(22) PCT Filed: Jan. 18, 2024
(86) PCT No.: PCT/US2024/012013

§ 371 Date: Jul. 15, 2025

Related U.S. Application Data
(60) Provisional application No. 63/440,175, filed on Jan. 20, 2023.

(51) Int. Cl. A61B 34/00; A61B 34/20; A61B 34/30; A61B 34/35; A61B 90/00

(52) U.S. Cl. CPC A61B 34/76; A61B 34/35; A61B 2034/2048; A61B 2034/302; A61B 2090/064; A61B 2090/067

(57) ABSTRACT

Systems and methods are provided for control of a surgical system. Accordingly, an output signal is received from a force sensor unit. The output signal corresponds to a sensed magnitude of forces exerted on a medical instrument and includes a vibratory component of the sensed magnitude and a commanded-motion component of the sensed magnitude. A vibratory component of the sensed magnitude that corresponds to a non-commanded motion of the medical instrument is determined. A modified force-magnitude signal is generated by filtering out the vibratory component from the sensed magnitude. The modified force-magnitude signal corresponds to a force resulting from a commanded motion of the medical instrument. A haptic feedback is provided to an input device of a surgical system based on the modified force-magnitude signal.

### CROSS-REFERENCE TO RELATED APPLICATIONS

This application claims priority to and the filing date benefit of U.S. Provisional Patent Application No. 63/440,175, entitled “SYSTEMS AND METHODS FOR CONTROL OF A SURGICAL SYSTEM,” filed Jan. 20, 2023, the disclosure of which is incorporated herein by reference in its entirety.

### BACKGROUND

The embodiments described herein relate to surgical systems, and more specifically to teleoperated surgical systems. More particularly, the embodiments described herein relate to systems and methods for identifying and filtering a vibratory component of a sensed force and generating a resultant modified force-magnitude signal used to control a surgical system that includes force feedback provided to a person operating the system.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). In a mechanically grounded telesurgical system, an instrument used to carry out a surgical action is supported by a kinematic chain. This kinematic chain typically includes a mechanically grounded base (e.g., resting on the floor, or mounted to the ceiling, a wall, or a portion of the operating table), a manipulator support structure that supports a teleoperated manipulator, and the manipulator to which the instrument is coupled.

Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient via a cannula to position the end effector at a work site within the patient's body. The clinician operating the telesurgical system controls one or more input devices so that motion of the input devices results in corresponding motion of the instrument as a whole, the end effector, or a portion of the end effector. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector, and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

In order to minimize forces applied to the body wall of the patient surrounding the incision or natural orifice, the instrument of the MIS system is positioned and manipulated in a manner such that a kinematic center of motion is maintained at the incision or orifice. The manipulator moves the instrument and the instrument's components into various kinematic poses to carry out a surgical action, and the center of motion may be constrained to remain at the desired location in space by the manipulator's physical design (a hardware-constrained center of motion) or by software controlling the manipulator's motion (a software-constrained center of motion). Since the manipulator moves the instrument and the constrained center of motion is physically remote from the manipulator itself, the center of motion of is called a remote center of motion (a “remote center”).

Force sensing surgical instruments are known and together with associated telesurgical systems may be used to provide haptic feedback to a surgeon operating the telesurgical system to perform a surgical procedure. The haptic feedback increases the immersion, realism, and intuitiveness of the surgeon's experience while performing the telesurgical procedure. For effective haptics rendering and accuracy, force sensors to sense forces between a teleoperated surgical instrument and tissue may be placed on the instrument at various locations, such as close to the anatomical tissue interaction (i.e., near the instrument's distal end, where the end effector is located). One representative design approach is to include a force sensor unit having electrical sensor elements (e.g., strain sensors or strain gauges) at the distal end of a medical instrument shaft to measure strain imparted to the medical instrument by the end effector's tissue interactions. The measured strain is used to determine the force imparted to the medical instrument and so as input upon which the desired haptic feedback to the operator is generated.

During telesurgical system operation, mechanical compliance in the kinematic chain between the mechanically grounded base of the manipulator unit supporting one or more teleoperated instrument manipulators and the location at which an instrument is mounted to its corresponding teleoperated manipulator can result in instrument vibration in up to all three Cartesian axes. Vibrations may originate in various ways, such as contact between the kinematic chain and an external object, or even inertia due to a manipulator's abrupt start and stop motions. If the instrument end effector is in free space (e.g., within a patient body cavity), then the effect of these vibrations on the force sensor unit is negligible, and the operator does not experience any haptic feedback as a result of the vibrations. But if a distal portion of the instrument is constrained in some way, such as the end effector being in contact with patient hard tissue (bone) or another instrument, or by grasping or retracting patient tissue, the constraint causes the vibrations to create corresponding reactive forces on the instrument. The force sensor unit senses these reactive forces, and as a result they are used to generate corresponding haptic feedback to the telesurgical system operator. This small, vibratory haptic feedback can distract or confuse the operator viewing the surgical site because there is no apparent reason for the haptic sensation. And at times, this vibratory haptic feedback may be incorrect for a situation, such as the operator perceiving the vibrations as the end effector moving over a rough surface when in fact the surface is smooth.

In view of the aforementioned, the art is continuously seeking new and improved systems and methods for control of a surgical system based on the accurate measurement of the forces imparted to the medical instrument.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

The systems and methods described herein facilitate the control of a MIS system. In order to minimize the forces applied to the body wall of the patient at the point of entry of a surgical instrument, the system establishes a kinematic remote center about which the instrument, and the cannula through which it is inserted, rotate, revolve, and/or pivot during an operation. The systems and methods described herein facilitate the accommodation of deviations between an actual location of the kinematic remote center and the location of kinematic remote center, as perceived by a controller of the system. Specifically, the systems and methods described herein facilitate the identification of a portion of an output signal of a force sensor unit of the instrument that is attributable to the effects of the deviation. The identified effects can then be filtered from the output signal of the force sensor and the modified output of the force sensor can be used to generate accurate force feedback and/or can support other functions of a surgical system.

In one aspect, the present disclosure is directed to a surgical system that includes a medical instrument. The medical instrument is supported by a manipulator unit that moves the instrument. A force sensor unit is coupled to the medical instrument to provide indications of forces applied to the instrument, such as at a distal end portion of the instrument. A user control unit that includes an input device can be operably coupled to the medical instrument and to the manipulator unit to allow an operator to move the medical instrument during a medical procedure. A controller is operably coupled to the manipulator unit, the input device, and the force sensor unit to provide a control relationship between these components. The controller includes at least one processor, a force-modifier module, and a haptic feedback module that during a medical procedure provides haptic feedback to the input device based on output from the force sensor unit. The controller is configured to perform a set of operations. The set of operations includes receiving an output signal from the force sensor unit. The output signal corresponds to a sensed magnitude of forces exerted on the medical instrument. The output signal includes a vibratory component of the sensed magnitude and a commanded-motion component of the sensed magnitude. The set of operations also includes determining, via the force-modifier module, the vibratory component corresponding to a non-commanded motion of the medical instrument. A modified force-magnitude signal is generated via the force-modifier module by filtering out the vibratory component. The modified force-magnitude signal corresponds to a force resulting from a commanded motion of the medical instrument. A haptic feedback based on the modified force-magnitude signal is provided via the haptic feedback module to the input device.

In some embodiments, the system includes a motion tracker operably coupled to the controller. The motion tracker is configured to measure a displacement of a portion of the medical instrument as expressed in a designated reference frame. The vibratory component of the sensed magnitude is correlated with the displacement of the portion of the medical instrument during a time interval.

In some embodiments, the manipulator unit includes an arm assembly. The arm assembly includes a spar supporting the medical instrument. The motion tracker includes an inertial measurement unit coupled to the spar. The displacement of the portion of the medical instrument is associated with both an acceleration output and an angular rate output of the inertial measurement unit.

In some embodiments, the non-commanded motion of the medical instrument is associated with a displacement of a portion of the medical instrument from a nominal position. The displacement during a time interval corresponds to a vibratory displacement of the portion of the medical instrument. The vibratory component of the sensed magnitude is determined based at least in part on the vibratory displacement of the portion of the medical instrument.

In some embodiments, the displacement of the portion of the medical instrument corresponds to displacement of a remote center of motion of the medical instrument.

In some embodiments, the system includes a motion tracker operably coupled to the controller, and the controller includes a displacement module. In such embodiments, the set of operations includes receiving, via the displacement module, one or more inputs from the motion tracker. The inputs are associated with the displacement of the portion of the medical instrument from the nominal position as expressed in a designated reference frame.

In some embodiments, the inputs include an indication of a linear acceleration vector and an indication of an angular velocity vector of the portion of the medical instrument.

In some embodiments, the manipulator unit includes an arm assembly. The arm assembly includes a spar supporting the medical instrument. The motion tracker is coupled to the spar at a mounting position. The mounting position is separated from the portion of the medical instrument. In such embodiments, the set of operations further includes determining a first linear velocity vector of the motion tracker at the mounting position based on the linear acceleration vector from the motion tracker. A modified angular velocity vector is generated by applying a position constant to the angular velocity vector of the motion tracker. The position constant correlates the angular velocity vector of the motion tracker to the angular velocity vector of the portion of the medical instrument. The first linear velocity vector is combined with the modified angular velocity vector to determine a second linear velocity vector of the portion of the medical instrument. The displacement of the portion of the medical instrument from the nominal position is determined based on the second linear velocity vector.

In some embodiments, the set of operations includes determining, via the force-modifier module, the vibratory component of the sensed magnitude based at least in part on the output signal and the displacement of the portion of the medical instrument from the nominal position.

In some embodiments, the set of operations includes applying, via the force-modifier module, a band-pass filter to the output signal to generate a filtered output signal. Applying, via the force-modifier module, the same band-pass filter to a signal indicative of the displacement of the portion of the medical instrument to generate a filtered displacement signal. The operations also include determining, via the force-modifier module, an adaptive gain based at least in part on the filtered output signal and the filtered displacement signal. The adaptive gain is combined with the filtered displacement signal to determine the vibratory component of the sensed magnitude.

In some embodiments, the band-pass filter is one of a set of band-pass filters. Each band-pass filter is associated with a specified frequency band of a set of specified frequency bands. Each specified frequency band is associated with a known vibratory displacement of the medical instrument, and the force-modifier module is configured to determine the vibratory component at each of the set of specified frequency bands.

In some embodiments, the non-commanded motion of the medical instrument results from a compliance of the manipulator unit.

In some embodiments, the manipulator unit includes an arm assembly supported by a base. A first specified frequency band corresponds to a sympathetic frequency of the arm assembly, and a second specified frequency band corresponds to a sympathetic frequency of the base.

In some embodiments, the set of operations includes determining a gain state of the haptic feedback module. Accordingly, filtering out of the vibratory component of the of the sensed magnitude includes applying a tuning gain, which is based at least in part on the gain state, to the vibratory component.

In one aspect, the present disclosure is directed to a method of control for a surgical system. The surgical system can include any of the features described herein. The method includes determining a coordinate location for a nominal position of a portion of the medical instrument. The coordinate location for the nominal position remains unchanged throughout a procedure. The method also includes receiving a linear acceleration vector from a motion tracker configured to monitor a motion of the portion of the medical instrument. Additionally, the method includes receiving an angular velocity vector from the motion tracker. A displacement module of the controller determines a displacement of the portion of the medical instrument relative to the nominal position based on the linear acceleration vector and the angular velocity vector. An operation of the surgical system is modified based on the displacement of the portion of the medical instrument from the nominal position.

In some embodiments, determining the displacement relative to the nominal position includes determining a displacement magnitude along three axes that are orthogonal to one another.

In some embodiments, the manipulator unit of the surgical system includes an arm assembly. The arm assembly includes a spar supporting the medical instrument. The motion tracker of the surgical system is an inertial measurement unit. The inertial measurement unit is coupled to the spar at a mounting position, and the mounting position is separated from the portion of the medical instrument.

In some embodiments, the displacement of the portion of the medical instrument corresponds to the displacement of a remote center of motion of the medical instrument relative to the nominal position of the remote center as determined by the controller.

In some embodiments, the manipulator unit includes an arm assembly. The arm assembly includes a spar supporting the medical instrument. The motion tracker is coupled to the spar at a mounting position. The mounting position is separated from the portion of the medical instrument, and the determining the displacement of the portion of the medical instrument includes determining a first linear velocity vector of the motion tracker at the mounting position based on the linear acceleration vector from the motion tracker. A modified angular velocity vector is generated by applying a position constant to the angular velocity vector from the motion tracker. The position constant correlates the angular velocity vector of the motion tracker to the angular velocity vector of the portion of the medical instrument. The first linear velocity vector is combined with the modified angular velocity vector to determine a second linear velocity vector of the portion of the medical instrument. The displacement of the portion of the medical instrument from the nominal position is determined based on the second linear velocity vector of the portion of the medical instrument.

In some embodiments, modifying the operation of the surgical system includes at least one of modifying a haptic feedback provided to the input device, initiating a calibration sequence, applying a damping factor to a commanded movement of the surgical system, halting an operation of the surgical system, generating an error signal, and generating a service request for the surgical system.

In one aspect, the present disclosure is directed to a method of control for a surgical system. The surgical system can include any of the features described herein. The method can include any of the operations described herein.

These and other features, aspects and advantages of the present invention will become better understood with reference to the following description and drawings.

### BRIEF DESCRIPTION OF THE DRAWINGS

FIG. 1A is a diagrammatic illustration of a portion of a known medical instrument depicting various positioning's of the instrument within a patient but not in contact with the target location.

FIG. 1B is a diagrammatic illustration of a portion of a known medical instrument depicting various positioning's of the instrument within a patient but not in contact with the target location.

FIG. 2 is a plan view of a minimally invasive teleoperated medical system according to an embodiment being used to perform a medical procedure such as surgery.

FIG. 3 is a perspective view of a user control console of the minimally invasive teleoperated surgery system shown in FIG. 2.

FIG. 4 is a perspective view of an optional auxiliary unit of the minimally invasive teleoperated surgery system shown in FIG. 2.

FIG. 5 is a front view of a manipulator unit, including a plurality of instruments, of the minimally invasive teleoperated surgery system shown in FIG. 2.

FIG. 6 is a perspective view of a medical instrument according to an embodiment.

FIG. 7 is a side view of a portion of the medical device of FIG. 6 with an outer shaft removed.

FIG. 8 is a perspective view of a cannula of the minimally invasive teleoperated surgery system shown in FIG. 2.

FIG. 9 is a perspective view of an arm assembly of the manipulator unit of FIG. 5 depicting a perceived position and an actual position according to an embodiment.

FIG. 10A is a flow chart of a set of operations for control of a surgical system.

FIG. 10B is a flow chart of a set of operations for control of a surgical system.

FIG. 10C is a flow chart of a set of operations for control of a surgical system.

FIG. 11A is a schematic diagram of a portion of a control system for control of a surgical system.

FIG. 11B is a schematic diagram of a portion of a control system for control of a surgical system.

FIG. 12 is a schematic illustration of a controller for use with a minimally invasive teleoperated surgery system according to an embodiment.

FIG. 13 is a flow chart of a method of control for a surgical system according to an embodiment.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of grasping, cutting, and manipulating operations associated with minimally invasive surgery. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that may rotate with reference to a proximal clevis (one DOF). Thus, in some embodiments, the medical instruments or devices of the present application may enable motion in six DOFs. The embodiments described herein further may be used to deliver haptic feedback to a system operator based on a load indication from the force sensor unit.

Generally, the present disclosure is directed to systems and methods for controlling a surgical system (system) such as a minimally invasive teleoperated surgery system. In particular, the present disclosure includes a system and methods that may facilitate the accurate sensing (e.g., measuring) of loads affecting a medical instrument and the delivery of haptic feedback based on the sensed loads. Accordingly, the systems and methods described herein facilitate the detection and accommodation of deviations of a portion (e.g., a remote center) of the instrument from a defined position that create corresponding reactive forces on the instrument. As used herein, the term “remote center” refers to a kinematic remote center about which an object (i.e., the cannula and the instrument inserted therein) can rotate, revolve, and/or pivot.

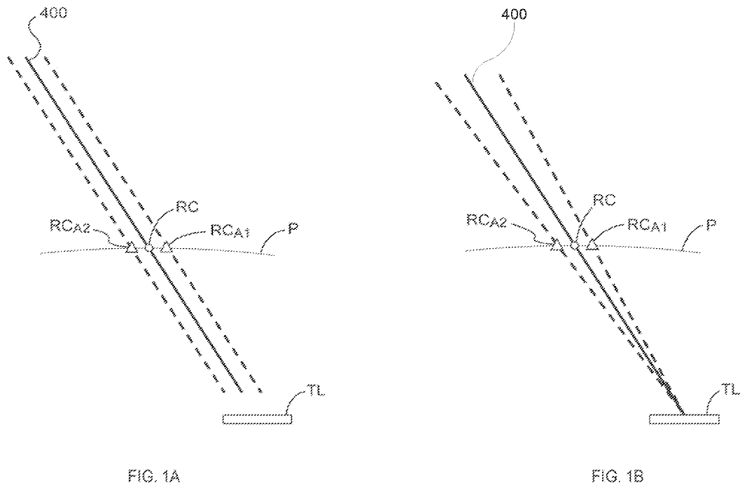

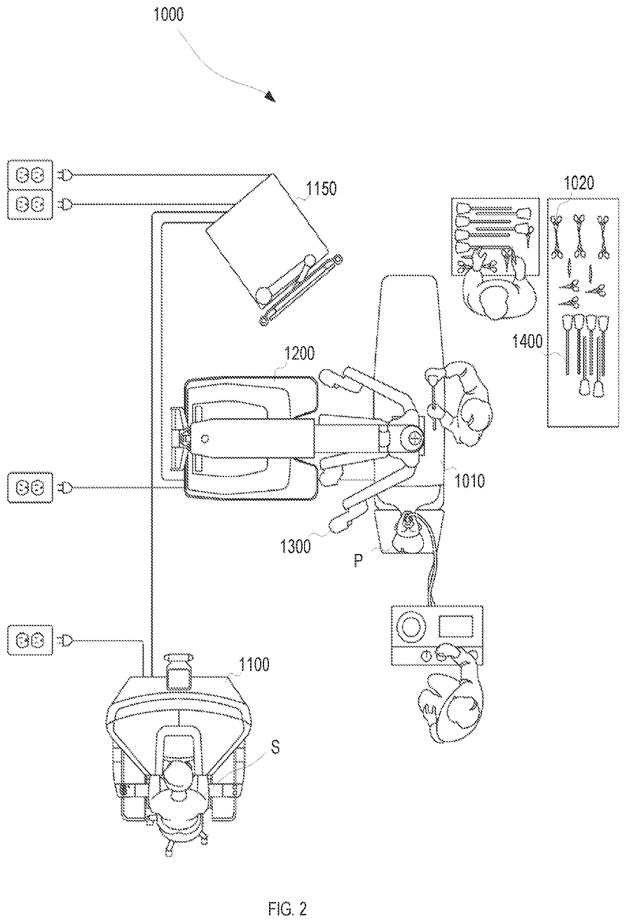

By way of illustration, in the kinematic chain from the mechanical ground to the end effector, vibrations can cause the instrument's actual remote center of motion to deviate slightly from the control system's defined center of motion. The remote center displacements can correspond to the instrument end effector's vibration, and so to the reactive forces sensed by the instrument's force sensor unit, and to the resulting unwanted haptic feedback to the operator. For example, as depicted in FIGS. 1A and 1B, which are diagrammatic views of a minimally invasive surgical instrument inserted though a patient's body wall to a surgical site, the controller presumes the remote center RC (e.g., the defined location of the remote center within the control system) is fixed at a known location in space (e.g., at the patient body wall P as shown). In practice, however, the mechanical compliance within the kinematic chain discussed above can result in small, deviations of the remote center (as illustrated by actual remote centers RC_{A1}, RC_{A2}, which depict a range of motion of the actual, physical remote center location in all three Cartesian dimensions) from the presumed fixed coordinates. In other words, the controller acts to control the instrument with the manipulator about a fixed point of intersection of the instrument 400 and the body wall the patient P (i.e., about the remote center RC), however a physical measurement of the actual point of intersection would reveal that the point of intersection may have deviated to an actual remote center RC_{A1} or RC_{A2}.

As depicted in FIG. 1A, the instrument 400 is not in contact with a target location TL within the patient. Therefore, small (sub-millimeter), vibratory deviations of the instrument (illustrated by the dashed lines showing there is no constraint at the instrument's distal end) result in corresponding small, vibratory remote center deviations between actual locations RC_{A1}, RC_{A2}. These deviations are not sensed by (i.e., do not have a significant impact on) an output of a force sensor unit configured to sense force acting on the distal end of the instrument.

As depicted in FIG. 1B, however, in some instances, the instrument 400 is in contact with the target location, and so the small, vibratory deviations of the instrument (illustrated by the dashed lines) result in corresponding small, vibratory remote center deviations between the remote center (as defined by the controller) and the actual location of the remote center RC_{A1} or RC_{A2}. This deviatio can result in corresponding vibratory forces sensed that are by the force sensor unit as a force affecting the instrument. As a result, the force indications resulting from the deviations of the of the remote center from the presumed remote center RC location can affect the haptic feedback provided to the operator. This vibratory haptic feedback may distract and confuse the operator because there is no apparent reason for it. Said another way, the vibratory deviations can be detected and/or measured by the force sensor unit as forces affecting the instrument 400, particularly when the instrument is in contact with a rigid structure (e.g., a bone or another instrument) or is otherwise constrained (e.g., by grasping, retracting, or palpating) other reasonably stiff tissue. Because the output of the force sensor is employed by the control system to generate haptic feedback, the inclusion of the forces resulting from the vibratory deviations can distort the desired haptic feedback. Similarly stated, if instrument 400 vibrates while its distal end is constrained, such as being in contact with tissue, the instrument's force sensor will sense the vibrations as reactive forces between the tissue and the instrument's distal end. As a result, these sensed vibratory reactive forces will be output to the system operator as incorrect haptic feedback. For example, the inclusion of the vibratory forces can result in the operator perceiving that the instrument is moving across a rough surface when the surface is actually smooth, or the operator perceiving vibration when grasping apparently stationary tissue. Joint position encoders are not able to sense vibration of the entire manipulator itself.

As described herein, the force sensor unit of the medical instrument is configured to generate output signals that correspond to a magnitude of the forces exerted on the medical instrument. For example, the output signal can correspond to the magnitude of the forces being applied by the medical instrument to a target location (e.g., gripping, cutting, probing, lifting, relocating, separating, pulling, etc.) and/or the magnitude of the forces applied to the medical instrument by the target location (e.g., the weight and resistance of the target location). However, the output signal can also include forces that result from the deviations the actual remote center location from the fixed remote center location (e.g., the remote center location) as perceived by the controller of the system, which is based on the positioning of elements of the structure supporting the instrument (i.e., an arm assembly of a manipulator unit of the system). In other words, the output signal can include the magnitudes of forces that result from a commanded motion of the instrument and those that result from a non-commanded motion of the instrument (e.g., vibratory deviations of the remote center from the fixed location), which can be detected by the force sensor unit as a force affecting the instrument. Accordingly, the systems and methods described herein determine the vibratory component of the output signal that corresponds to the non-commanded motion (e.g., result from the vibratory deviations of the actual remote center). Once identified, the vibratory component can be filtered from the output signal to generate a modified force-magnitude signal. A haptic feedback can then be provided to the input device of the system based on the modified force-magnitude signal.

Insofar as the vibratory component of the output signal can be attributed to deviations of the actual location of the remote center from the fixed nominal location of the remote center (e.g., the location of the fixed remote center), determining the magnitude of the deviations of the remote center location can facilitate the identification and mitigation of the vibratory component of the output signal. To that end, the system can include a motion tracker that is configured to measure the displacement of a portion of the medical instrument. The motion tracker can, for example, be an inertial measurement unit (IMU), an optical tracker, a laser measuring device, and/or other similar system. In some embodiments, the displacement of the portion of the medical instrument corresponds to the displacement of the remote center. However, in some embodiments, the displacement of the portion of the medical management can correspond to the displacement of an additional portion of the medical instrument from which the displacement of the remote center from the nominal position can be derived. For example, an acceleration output and an angular rate output of the IMU can correspond to the motion of the IMU itself but can be transformed to determine the displacement of the remote center.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms-such as “beneath”, “below”, “lower”, “above”, “upper”, “proximal”, “distal”, and the like-may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation defines the body's pose (e.g., a kinematic pose).

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

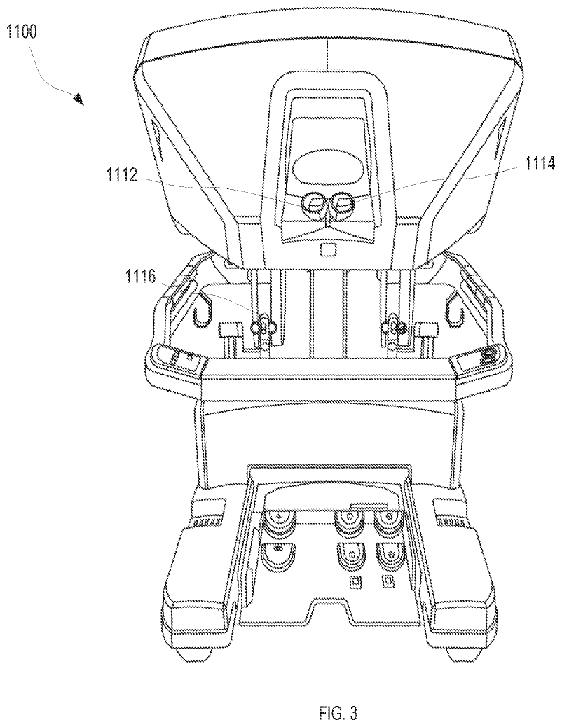

FIG. 2 is a plan view illustration of a teleoperated surgical system (“system”)1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by an operator of the system, such as a surgeon or other skilled clinician S, during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled medical instrument (instrument) 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the system 1000.

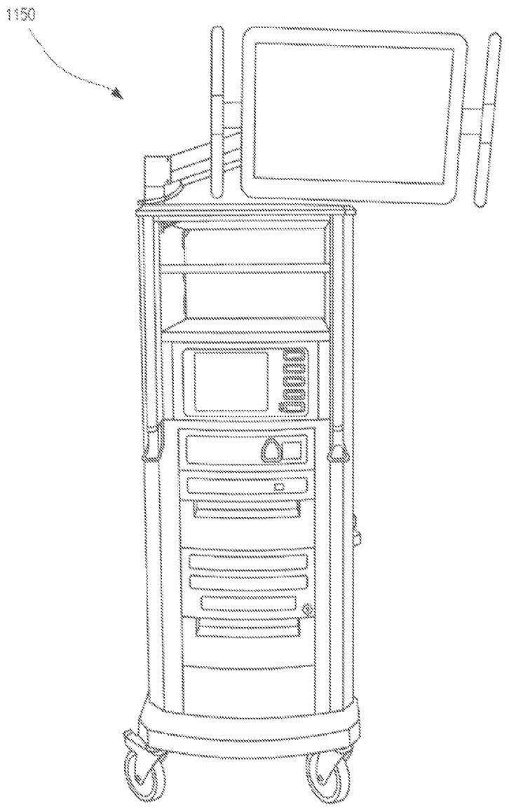

FIG. 3 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116 (input device), which in turn causes the manipulator unit 1200 (shown in FIG. 2) to manipulate one or more tools. The input devices 1116 provide at least the same degrees of freedom as instruments 1400 with which they are associated to provide the surgeon S with telepresence, or the perception that the input devices 1116 are integral with (or are directly connected to) the instruments 1400. In this manner, the user control unit 1100 provides the surgeon S with a strong sense of directly controlling the instruments 1400. To this end, position, force, strain, or tactile feedback (not shown) or any combination of such feedback from the instruments 1400 are provided back to the surgeon's hand or hands through the one or more input devices 1116.

The user control unit 1100 is shown in FIG. 2 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

FIG. 4 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the auxiliary equipment unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

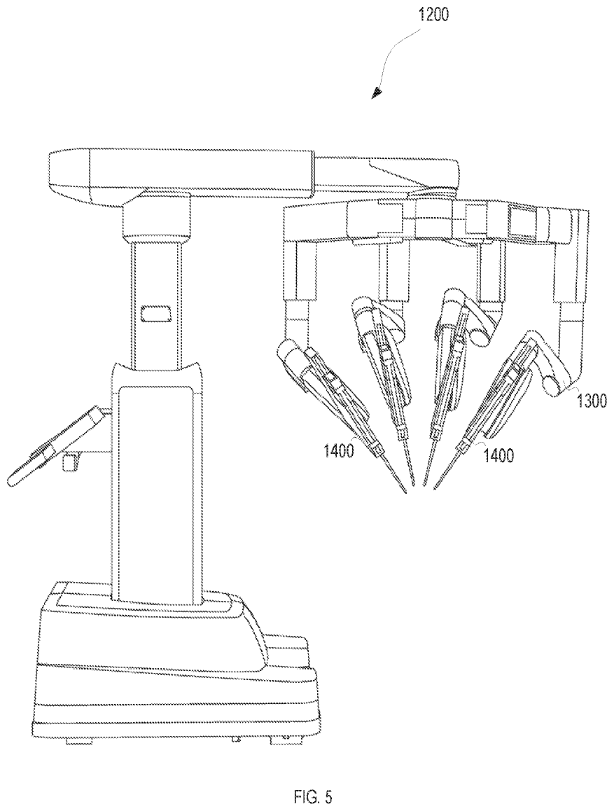

FIG. 5 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

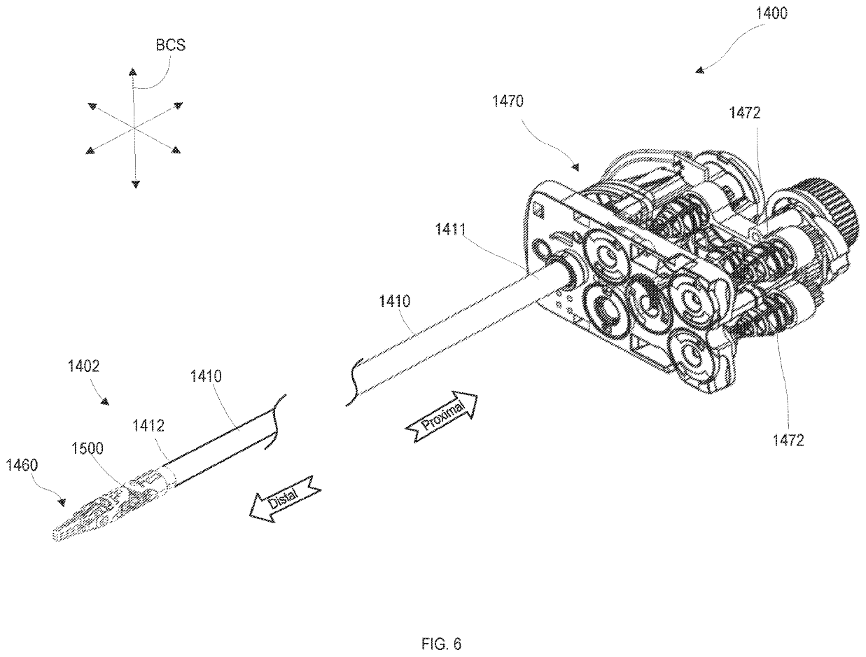

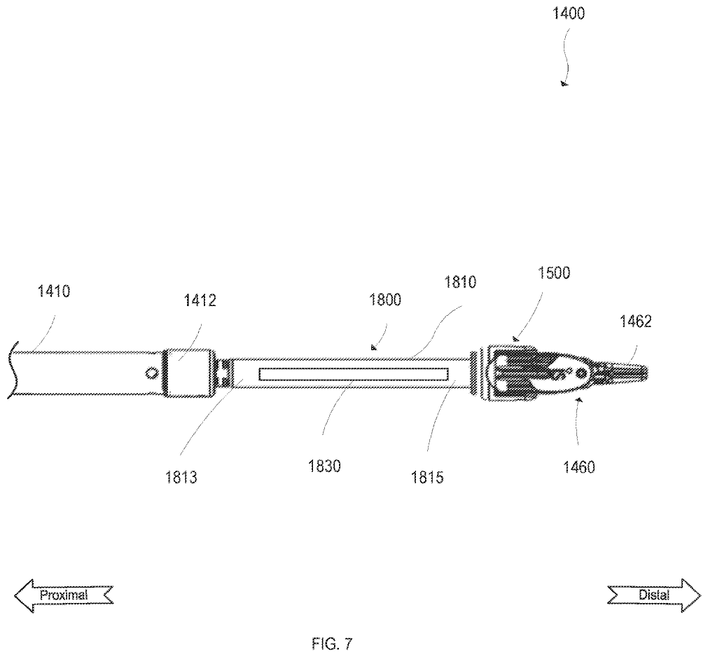

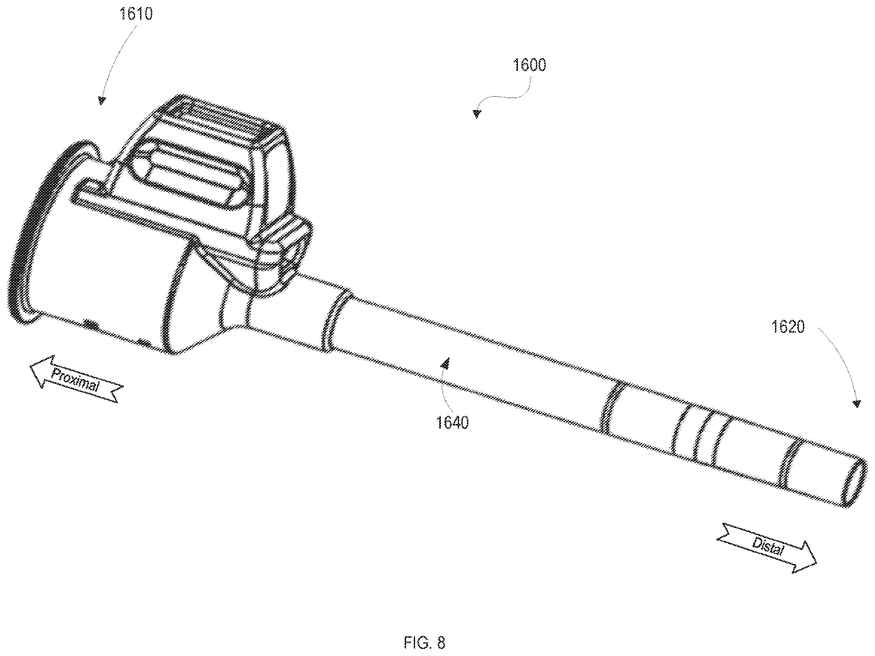

Referring now to FIGS. 6 and 7, a perspective view of the instrument 1400 is depicted in FIG. 6, and a side view of a portion of the instrument 1400 with an outer shaft portion removed is depicted in FIG. 7. In some embodiments, the instrument 1400 or any of the components therein are optionally parts of a surgical system that performs surgical procedures, and which can include a manipulator unit, a series of kinematic linkages, a set of cannulas, or the like. The instrument 1400 (and any of the instruments described herein) can be used in any suitable surgical system, such as the MIRS system 1000 shown and described above. As shown in FIG. 6, the instrument 1400 includes a proximal mechanical structure 1470, a shaft 1410, a distal end portion 1402, and a set of cables (not shown). The cables function as tension elements that couple the proximal mechanical structure 1470 to the distal end portion 1402. In some embodiments, the distal end portion 1402 includes a distal wrist assembly 1500 and a distal end effector 1460. The instrument 1400 is configured such that movement of one or more of the cables produces movement of the end effector 1460 (e.g., pitch, yaw, or grip) about axes of a beam coordinate system BCS.

Moreover, although the proximal mechanical structure 1470 is shown as including capstans 1472, in other embodiments, a mechanical structure can include one or more linear actuators that produce translation (linear motion) of a portion of the cables. Such proximal mechanical structures can include, for example, a gimbal, a lever, or any other suitable mechanism to directly pull (or release) an end portion of any of the cables. For example, in some embodiments, the proximal mechanical structure 1470 can include any of the proximal mechanical structures or components described in U.S. Patent Application Pub. No. US 2015/0047454 A1 (filed Aug. 15, 2014), entitled “Lever Actuated Gimbal Plate,” or U.S. Patent No. U.S. Pat. No. 6,817,974 B2 (filed Jun. 28, 2001), entitled “Surgical Tool Having Positively Positionable Tendon-Actuated Multi-Disc Wrist Joint,” each of which is incorporated herein by reference in its entirety.

The shaft 1410 can be any suitable elongated shaft that is coupled to the wrist assembly 1500 and to the proximal mechanical structure 1470. Specifically, the shaft 1410 includes a proximal end 1411 that is coupled to the proximal mechanical structure 1470, and a distal end portion 1412 that is coupled to the wrist assembly 1500 (e.g., a proximal link of the wrist assembly 1500). The shaft 1410 defines a passageway or series of passageways through which the cables and other components (e.g., electrical wires, ground wires, or the like) can be routed from the proximal mechanical structure 1470 to the wrist assembly 1500. In some embodiments, the shaft 1410 can be formed, at least in part with, for example, an electrically conductive material such as stainless steel. In such embodiments, the shaft may include any of an inner insulative cover or an outer insulative cover. Thus, the shaft 1410 can be a shaft assembly that includes multiple different components. For example, the shaft 1410 can include (or be coupled to) a spacer that provides the desired fluid seals, electrical isolation features, and any other desired components for coupling the wrist assembly 1500 to the shaft 1410. Similarly stated, although the wrist assembly 1500 (and other wrist assemblies or links described herein) are described as being coupled to the shaft 1410, it is understood that any of the wrist assemblies or links described herein can be coupled to the shaft via any suitable intermediate structure, such as a spacer and a cable guide, or the like.

As depicted in FIG. 7, the instrument 1400 (e.g., the surgical or medical instrument) includes a force sensor unit 1800 including a beam 1810, with one or more strain sensors 1830. The strain sensor 1830 can include a set of strain gauges (e.g., tension strain gauge resistor(s) or compression strain gauge resistor(s)) arranged as at least one bridge circuit (e.g., Wheatstone bridges) mounted on a surface along the beam 1810. In some embodiments, the end effector 1460 can be coupled at a distal end portion 1815 of the beam 1810 (e.g., at a distal end portion 1402 of the instrument 1400) via the wrist assembly 1500. The shaft 1410 includes a distal end portion 1412 that is coupled to a proximal end portion 1813 of the beam 1810. In some embodiments, the distal end portion 1412 of the shaft 1410 is coupled to the proximal end portion 1813 of the beam 1810 via another coupling component (such as an anchor or coupler, not shown). In some embodiments, the force sensor unit 1800 can include any of the structures or components described in U.S. Patent Application Pub. No. US 2020/0278265 A1 (filed May. 13, 2020), entitled “Split Bridge Circuit Force Sensor,” which is incorporated herein by reference in its entirety.

In some embodiments, the end effector 1460 can include at least one tool member 1462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 1460 may be operatively coupled to the proximal mechanical structure 1470 such that the tool member 1462 rotates relative to shaft 1410. In this manner, the contact portion of the tool member 1462 can be actuated to engage or manipulate a target tissue during a surgical procedure. The tool member 1462 (or any of the tool members described herein) can be any suitable medical tool member. Moreover, although only one tool member 1462 is identified, as shown, the instrument 1400 can include two tool members that cooperatively perform gripping or shearing functions. In other embodiments, an end effector can include more than two tool members.

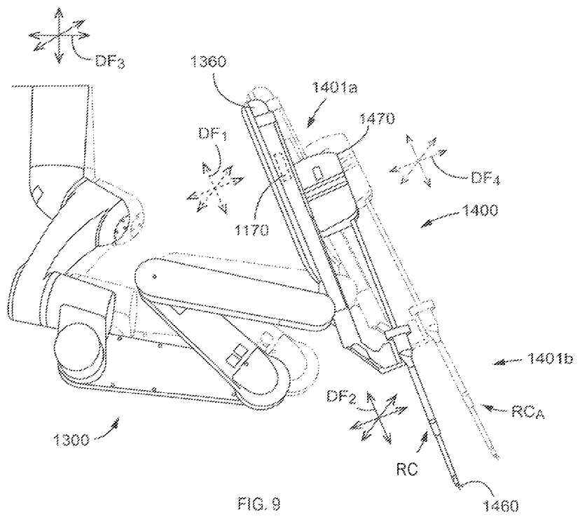

FIG. 8 depicts a perspective view of a cannula 1600 for use with the system 1000. As depicted, the cannula 1600 can be configured to be coupled to the arm assembly 1300 and surround at least a portion of the instrument 1400 to facilitate access of the surgical site by the end effector 1460. Accordingly, the cannula 1600 can have a proximal end 1610 and a distal end 1620. A central channel 1640 extends between the proximal and distal ends 1610, 1620. As such, the cannula 1600 forms a channel or passage through which the instrument 1400 can be inserted to access the surgical site. As depicted, the cannula 1600 can be a straight cannula. However, in additional embodiments, the cannula 1600 can, for example, be a curved cannula having a combination of linear and nonlinear sections, a cannula with multiple non-parallel linear sections, a cannula with multiple curve sections having different characters, and/or a cannula with other combinations of linear and nonlinear sections. In some embodiments, the distal end 1620 of the cannula 1600 is inserted through an incision and into a body cavity of the patient. The proximal end 1610 of the cannula 1600 is maintained external to a body wall of the patient and is coupled to the arm assembly 1300 of the system 1000.

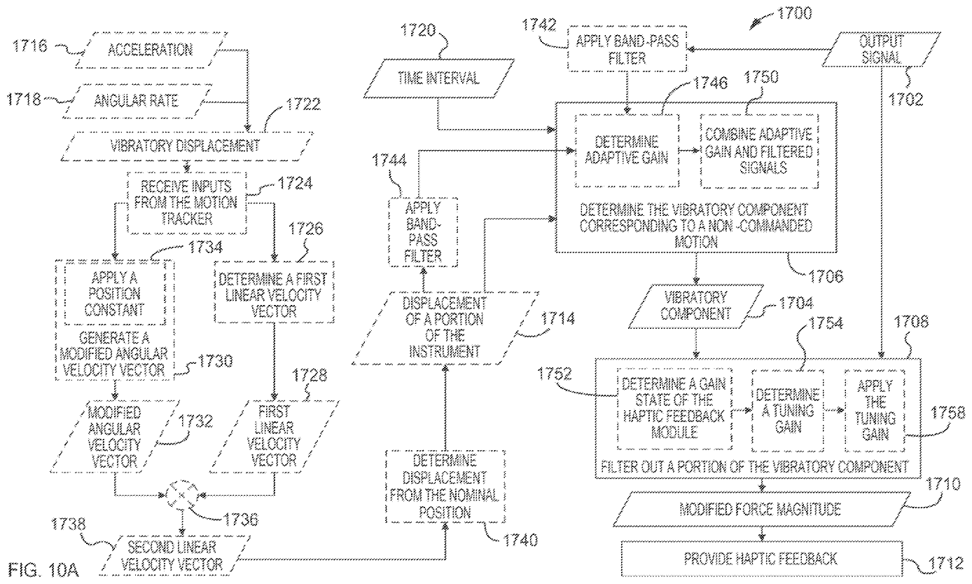

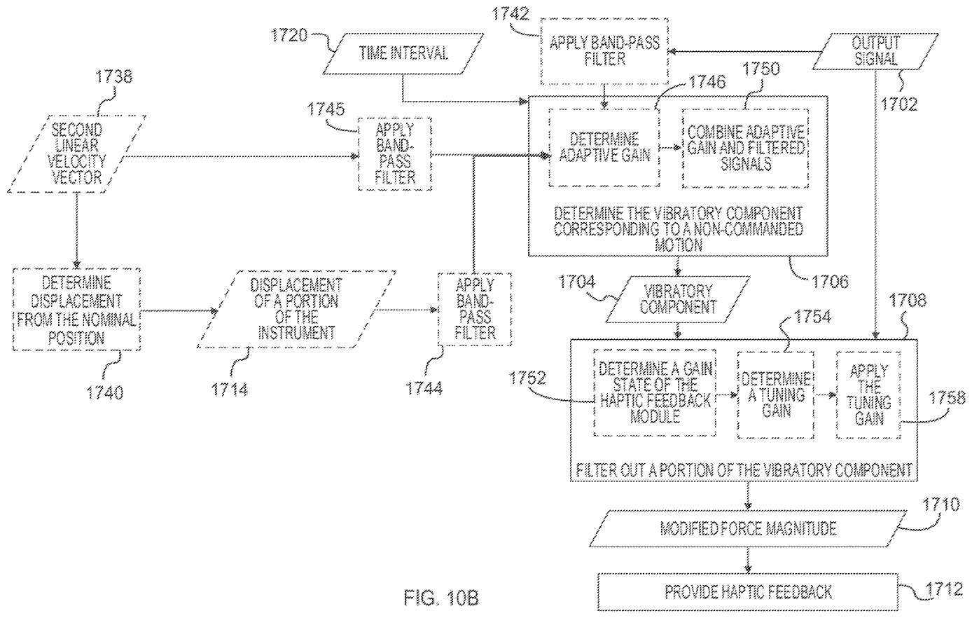

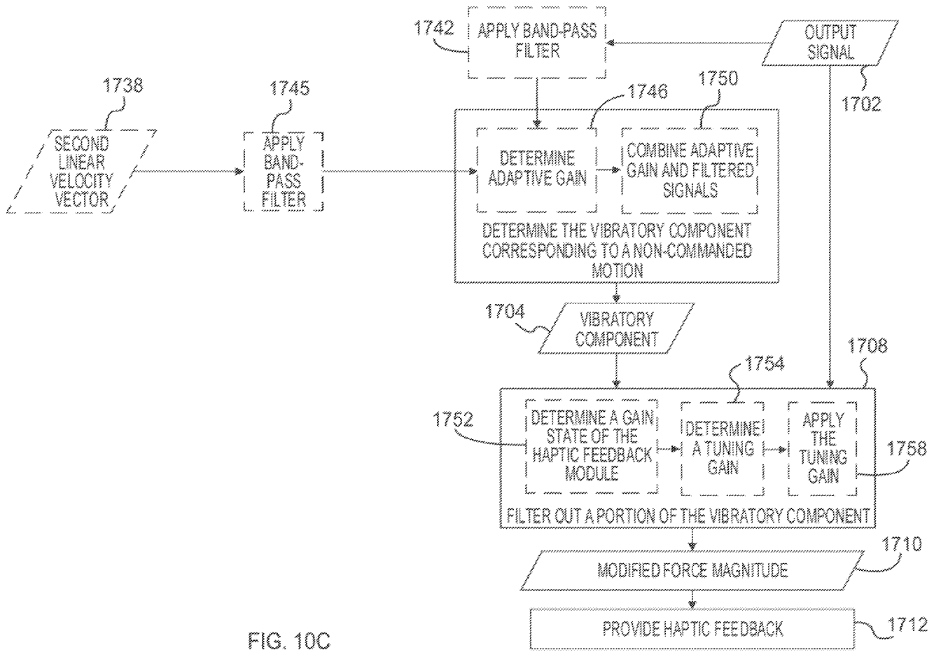

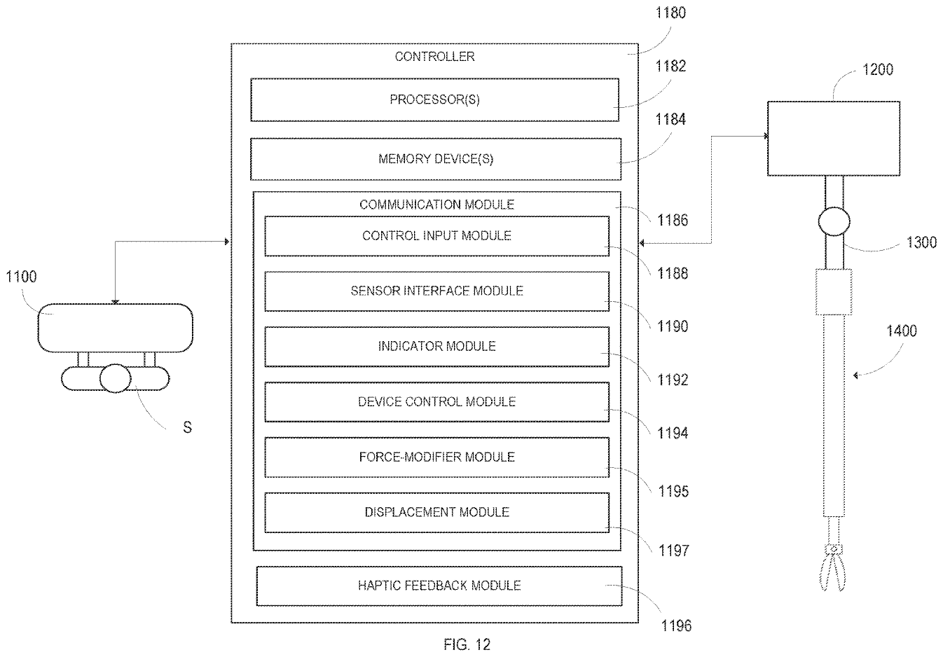

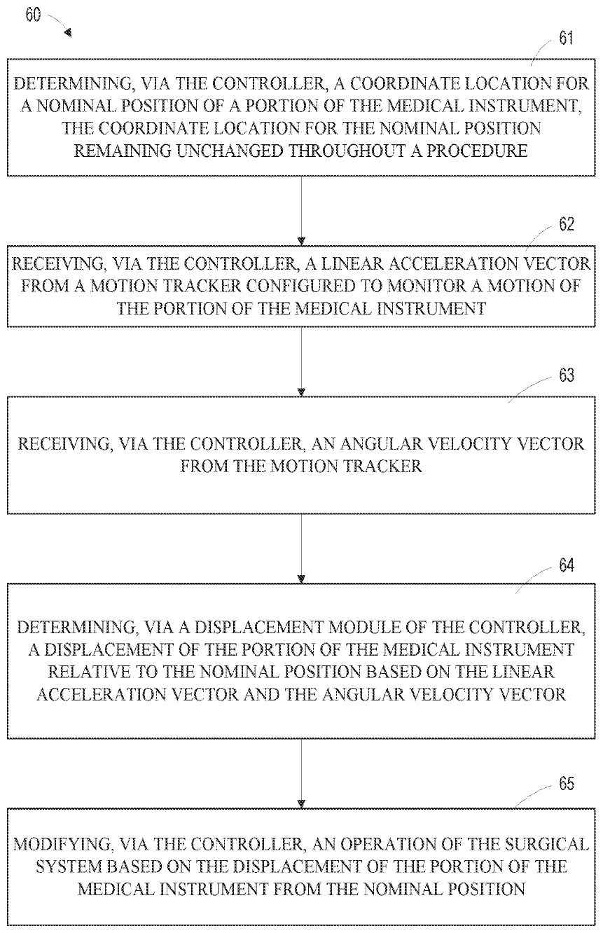

Referring now to FIGS. 9-12, in some embodiments, the system 1000 includes the force sensor unit 1800 that is coupled to the instrument 1400 that is supported by the manipulator unit 1200. The input device 1116 is operably coupled to the instrument 1400 and the manipulator unit 1200 as previously described. The system 1000 includes a controller 1180 (FIG. 12) that is operably coupled to the manipulator unit 1200, the input device 1116, and the force sensor unit 1800. The controller 1180 includes at least one processor 1182, a force-modifier module 1195, and a haptic feedback module 1196. In some embodiments, the controller 1180 also includes a displacement module 1197. The controller 1180 is configured to perform a set of operations 1700, such as those depicted in FIG. 10A. Although the set of operation 1700 is described with respect to the instrument 1400 and the controller 1180, in other embodiments, the set of operations 1700 and any of the methods described herein can be performed on any suitable instrument or any suitable controller.

In some embodiments, the set of operations 1700 includes receiving an output signal 1702 from the force sensor unit 1800. The output signal 1702 corresponds to a sensed magnitude of the forces exerted on the instrument 1400. The output signal 1702 can, for example, be an output of the strain sensor 1830 that is indicative of a strain magnitude developed by the beam 1810 in response to a load applied to the instrument 1400. Accordingly, the output signal 1702 can be a voltage differential measured across a Wheatstone bridge circuit.

The sensed magnitude indicated by the output signal 1702 can include a commanded-motion component that is associated with a commanded motion and a vibratory component 1704 that is associated with a non-commanded motion. The commanded-motion component can, for example, be a force that results from an input received from the input device 1116. In other words, the commanded-motion component can be the portion of the output signal 1702 that corresponds to a commanded surgical motion intended to achieve an objective of the operation, such as grasping a tissue sample or other similar operation. The non-commanded motion can, for example, be an unintended vibratory motion of the instrument 1400. Said another way, during certain operations, the instrument 1400 can develop or be subjected to an unintended vibratory motion. When the end effector 1460 is not in contact with a portion of the patient's body, the end effector 1460 can vibrate freely and the vibratory motion may not affect the output signal 1702. However, when the end effector 1460 is in contact with the target location, motion of the end effector 1460 can be restricted and the vibrations can result in a deflection of the beam 1810, which is then sensed by the force sensor unit 1800 as a load affecting the instrument 1400.

Since control of the system 1000 can be based, at least in part, on the output signal 1702, it should be appreciated that it may be desirable to mitigate the effects of the vibratory component 1704 of the output signal 1702. Accordingly, as depicted in FIG. 10A at 1706, in some embodiments, the set of operations 1700 includes employing the force-modifier module 1195 to determine the vibratory component 1704 of the output signal 1702 as described more fully below. The vibratory component 1704 corresponds to the non-commanded motion of the instrument 1400. As depicted at 1708, the force-modifier module 1195 can be employed to filter out the vibratory component 1704 to generate a modified force-magnitude signal 1710. The modified force-magnitude signal 1710 corresponds to a force resulting from the commanded motion of the instrument 1400. In some embodiments wherein a portion of the vibratory component 1704 is filtered from the output signal 1702, the modified force-magnitude signal 1710 be indicative of the force resulting from the commanded motion and a percentage of the force resulting from the non-commanded motion of the instrument 1400. However, in some embodiments wherein substantially the entirety of the vibratory component 1704 is filtered from the output signal 1702, the modified force-magnitude signal 1710 can substantially equate to the force resulting from the commanded motion. As depicted at 1712, control of the system 1000 (including the production of the haptic feedback) can be based, at least in part, on the modified force-magnitude signal 1710. For example, in some embodiments, the haptic feedback module 1196 can be employed to provide a haptic feedback to the input device 1116 based on the modified force-magnitude signal 1710. In other words, the haptic feedback provided to the input device 1116 can be based substantially on those forces affecting the instrument 1400 that result from the commanded motion of the instrument 1400 with the effects of the forces resulting from the non-commanded motion of the instrument 1400 being mitigated.

In some embodiments, the vibratory component 1704 of the sensed magnitude indicated by the output signal 1702 is correlated with the displacement 1714 of a portion 1401 of the instrument 1400 during a specified time interval. In some embodiments, the vibratory component 1704 of the sensed magnitude indicated by the output signal 1702 is correlated with the velocity (i.e., as indicated by a second linear velocity vector 1738) of a portion 1401 of the instrument 1400. In some embodiments, the vibratory component 1704 of the sensed magnitude indicated by the output signal 1702 is correlated with both the displacement 1714 and the velocity of a portion 1401 of the instrument 1400. Accordingly, in some embodiments, the system 1000 includes a motion tracker 1170. The motion tracker 1170 is operably coupled to the controller 1180. The motion tracker 1170 can, for example, be an inertial measurement unit (IMU), an optical tracker, a laser measuring device, and/or other similar system. The motion tracker 1170 is configured to measure a displacement of the portion 1401 of the instrument 1400. The displacement of the portion 1401 can be expressed in a designated reference frame DF₁ as depicted in FIG. 9. The designated reference frame DF₁ can be a coordinate reference frame centered at a starting, designated, and/or nominal position of the portion 1401 of the instrument 1400. As such, the displacement of the portion 1401 can be expressed as a displacement relative to the starting, designated, and/or nominal position of the portion 1401.

In FIG. 9, the solid lines depict the position of the arm assembly 1300 and instrument 1400 as perceived by the controller 1180, while the dashed lines depict the actual position of the arm assembly and instrument 1400 at a specified instant. In some instances, the difference between the perceived position of the instrument 1400 and the actual position of the instrument 1400 can be caused by vibratory motions due to the compliance of the arm assembly 1300. In some embodiments, the non-commanded motion of the instrument 1400 is associated with the displacement 1714 of the portion 1401 of the instrument 1400 from a nominal position (e.g., the position indicated by the solid lines in FIG. 9). The displacement 1714, such as depicted in FIG. 9, that occurs during a time interval 1720, can correspond to a vibratory displacement 1722 of the portion 1401 of the instrument 1400 that manifests as a deflection of the beam 1810. As such, the vibratory component 1704 of the sensed magnitude indicated by the output signal 1702 is determined based, at least in part, on the vibratory displacement 1722 of the portion 1401 of the instrument 1400.

As further depicted in FIG. 9, in some embodiments, the displacement of the instrument 1400 corresponds to displacement of the remote center as indicated by portion 1401b. The remote center is a kinematic remote center about which the cannula and the instrument inserted therein can rotate, revolve, and/or pivot. The remote center can, for example, be defined at the intersection of the instrument and the body wall of the patient through which it is inserted. By rotating about the remote center, as opposed to other possible rotational centers, the movement of the instrument and the cannula, at the interface of the cannula and the patient's body is minimized. Once established at a designated fixed location (e.g., an initial location), the coordinates of the remote center within the memory device(s) 1184 of the controller 1180 remain fixed throughout the procedure as a remote center RC. Said another way, the remote center RC is the location of the kinematic remote center as calculated and understood by the controller 1180. Accordingly, the controller 1180 determines the location of the remote center RC based on inputs from encoders in various joints of the arm assembly 1300 that indicate the positioning of the linkages of the arm assembly 1300 at any given instant. The controller 1180 positions the components of the arm assembly 1300 to maintain the positioning of the remote center RC at the designated fixed location. The components of the arm assembly 1300 or manipulated by the controller 1180 such that the end effector 1460 is manipulated to achieve the desired effect while the remote center RC is maintained at the designated fixed location. However, as previously described, compliance within the arm assembly 1300 can result in deviations of the location of the actual remote center RCA (e.g., the physical location of the remote center during an operation) from the remote center RC.

As depicted at 1724, in some embodiments the controller 1180 is configured to receive one or more inputs (e.g., an acceleration output 1716 and/or an angular rate output 1718) from the motion tracker 1170, via the displacement module 1197. The inputs are associated with the displacement of the portion 1401 of the instrument 1400 from a nominal position (e.g., a designated position as perceived by the controller 1180). The inputs can describe the displacement in a designated reference frame DF. In some embodiments, the designated reference frame DF can be centered at the designated position of the motion tracker 1170 as depicted in FIG. 9 by reference frame DF₁. In some embodiments, the designated reference frame DF can be centered at the remote center RC as depicted in FIG. 9 by reference frame DF₂. However, in some embodiments, the designated reference frame DF can be centered at a designated location of the manipulator unit 1200 as depicted in FIG. 9 by reference frame DF₃, which can refer to a base frame. Similarly, reference frame DF₄ is indicative of a designated reference frame centered at a point of coupling between the proximal mechanical structure 1470 and the spar 1360 (e.g., a carriage frame). It should be appreciated that additional designated reference frames DF can be used and centered about different portions of the system 1000. It should further be appreciated that the various vectors and transforms described herein can be accomplished in and between any of the designated reference frames DF.

As depicted in FIG. 9, the arm assembly 1300 includes a spar 1360 that supports the instrument 1400. In some embodiments, the motion tracker 1170 is an IMU coupled to the spar 1360, and the portion 1401 of the instrument corresponds to the coupling location of the IMU as indicated by portion 1401a. In such embodiments, the designated reference frame DF can be an IMU frame (e.g., designated reference frame DF₁) centered at the perceived designated position of the IMU. Said another way, the IMU frame can be centered at the commanded position of the IMU when a commanded position and commanded orientation of the spar 1360 coincide with an actual position and orientation of the spar 1360 (i.e., when the perceived positions of the arm assembly 1300 and the instrument 1400 align with the actual positions of the same).

The IMU can include one or more accelerometers and one or more gyroscopes. In some embodiments, the IMU can include one accelerometer and one gyroscope for each of three principal orthogonal axes, such as the axes of the designated reference frame DF₁. The output of the accelerometer(s) can be expressed as a linear acceleration vector, while the output of the gyroscope(s) can be expressed as an angular rate. Accordingly, the displacement of the portion 1401a of the medical instrument is associated with both the acceleration output 1716 (i.e., the linear acceleration vector) and the angular rate (e.g., a rotational rate) output 1718 of the IMU. As depicted in FIG. 9, the acceleration output 1716 and angular rate output 1718 can be expressed in the designated reference frame DF₁ (i.e., the IMU frame).

In some embodiments, the mounting position (e.g., coupling location) of the motion tracker 1170 is separated from the portion 1401 of the instrument 1400. The outputs of the motion tracker 1170 can therefore be indicative of a movement of the mounting position (e.g., the movement of a portion of the arm assembly 1300 to which the motion tracker 1170 is coupled). For example, as depicted in FIG. 9, the mounting position of the motion tracker 1170 can be separated from the portion 1401b of the instrument 1400 corresponding to the remote center RC and the outputs of the motion tracker 1170 can be indicative of a movement of the spar 1360 at the mounting location of the motion tracker 1170 (e.g., the IMU). As such, it may be desirable to transform the outputs of the motion tracker 1170 to be indicative of a movement of a separate portion 1401b of the instrument 1400.

To transform the outputs of the motion tracker 1170 to be indicative of the movement of a separate portion 1401b of the instrument 1400, the controller 1180 can be configured to determine a first linear velocity vector 1728 at 1726. In some embodiments, the first linear velocity vector 1728 can be based on a linear acceleration vector (e.g., the acceleration output 1716) from the motion tracker 1170. For example, the acceleration output 1716 can be integrated to determine the first linear velocity vector 1728. At 1730, the controller 1180 can also be configured to generate a modified angular velocity vector 1732. As depicted at 1734, the modified angular velocity vector 1732 can be generated by applying a position constant to the angular velocity vector (e.g., the angular rate output 1718) of the motion tracker 1170. The position constant can, for example, be a position vector of the actual remote center RCA relative to the mounting position of the motion tracker 1170. For example, the position constant can be indicative of a separation distance between the mounting position of the motion tracker 1170 and the portion 1401b of the instrument 1400. As such, the position constant can be used to correlate the angular velocity vector of the motion tracker 1170 to an angular velocity of the portion 1401b of the instrument 1400 that is physically separated from the mounting position of the motion tracker 1170. Said another way, the modification of the angular velocity vector output of the motion tracker 1170 can transform the angular velocity output to be descriptive of an angular motion of the portion 1401b that is spaced apart from the mounting location of the motion tracker 1170.

As depicted at 1736 in FIG. 10A, the first linear velocity vector 1728 can be combined with the modified angular velocity vector 1732 to generate a second linear velocity vector 1738. The second linear velocity vector 1738 can be descriptive of a motion of the portion 1401b (e.g., the actual remote center RCA) of the instrument 1400. Said another way, the combination of the first linear velocity vector 1728 and the modified angular velocity vector 1732 can be descriptive of the motion of the portion 1401b when transformed from the inputs from the motion tracker 1170. Accordingly, at 1740, the controller 1180 is configured to determine the displacement 1714 of the portion 1401 of the instrument 1400 from the nominal position (e.g., the designated position) based on the second linear velocity vector 1738. For example, the controller 1180 can integrate the second linear velocity vector 1738 to determine the displacement 1714 of the actual remote center RCA relative to the remote center RC in the designated reference frame DF.

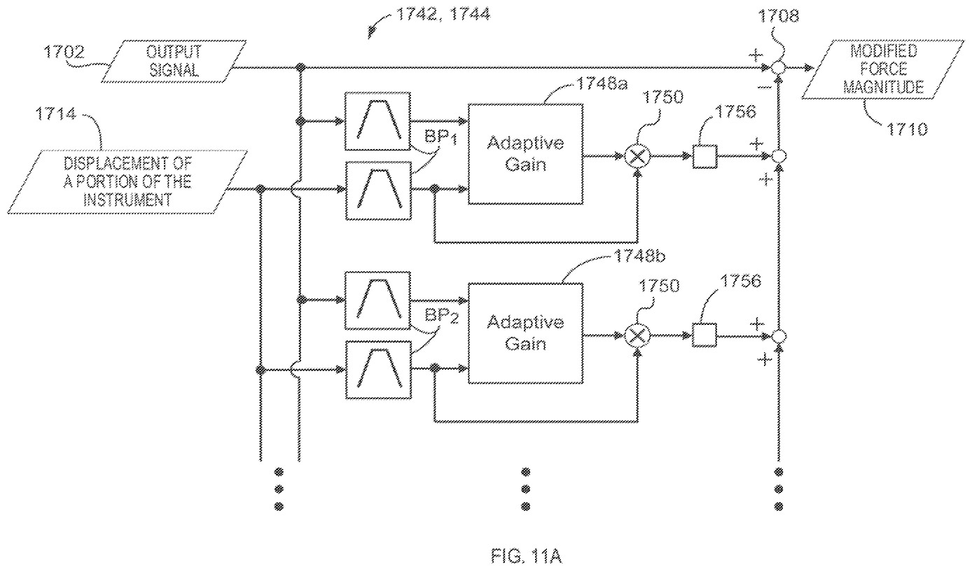

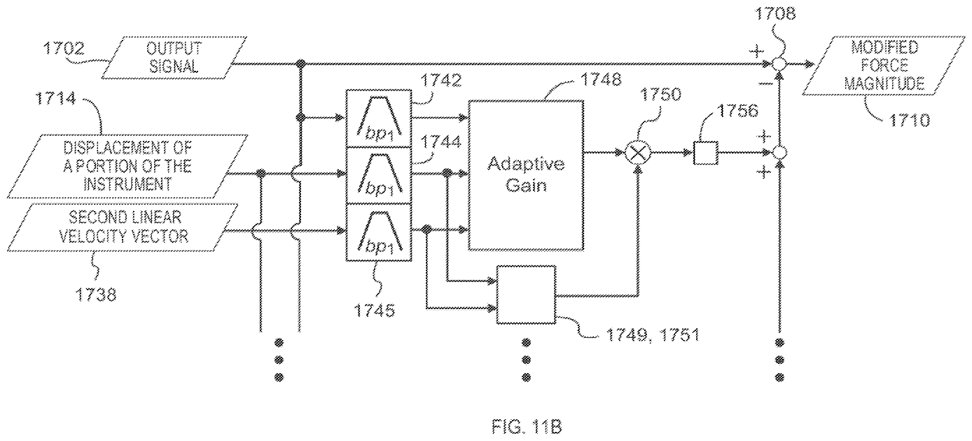

In some embodiments, the controller 1180 is configured to determine the vibratory component 1704 of the sensed magnitude of forces affecting the instrument 1400 based, at least in part, on the output signal 1702 and the displacement 1714 of the portion 1401 from the nominal position (e.g., the designated position). For example, the controller 1180 is configured to determine the vibratory component 1704 of the sensed magnitude based on the output signal 1702 and the displacement 1714, as determined from the second linear velocity vector 1738, of the actual remote center RCA relative to the remote center RC in the designated reference frame DF. In order to determine the vibratory component 1704, in some embodiments, the controller 1180 can be configured, via the force-modifier module 1195, to filter portions of the output signal 1702 that are indicative of the displacement of the portion 1401 of the instrument 1400. Accordingly, as depicted at 1742 in FIGS. 10A and 11A, the controller 1180 is configured to apply a band-pass filter BP to the output signal 1702 to generate a filtered output signal. At 1744, the same band-pass filter BP is also applied to the signal indicative of the displacement 1714 of the portion 1401 of the instrument 1400 to generate a filtered displacement signal.

In some embodiments, the band-pass filter BP is one of a set of band-pass filters (e.g., BP₁, BP₂, . . . BP_{N}). Each band-pass filter BP₁, BP₂ is associated with a separate frequency band. Each of the frequency bands is associated with a known vibratory displacement of the instrument 1400. For example, a first band-pass filter BPI can be associated with a first known frequency band associated with a force and a displacement along a first principal axis (e.g., along one of three orthogonal axis of a designated reference frame DF). Similarly, a second band-pass filter BP₂ can be associated with a second known frequency band associated with a force and a displacement along the same principal axis. Accordingly, the controller 1180 can apply a separate band-pass filter BP for each frequency band of a set of specified frequency bands along each principal axis. Therefore, the controller 1180, via the force-modifier module 1195, can facilitate the determination of the vibratory component 1704 at each of the specified frequency bands and along each of the principal axes in real time, during an operation.

In some embodiments, the frequency bands (e.g., frequencies associated with the non-commanded motion of the instrument 1400) can be determined based on historical data and/or experimentation. For example, in some embodiments, the end effector 1460 is brought into contact with a rigid body, a displacement of the portion 1401 of the instrument 1400 is developed (e.g., an instantaneous force applied to the instrument 1400 and/or the arm assembly 1300), and the magnitude of the output signal 1702 of the force sensor unit 1800 is recorded across a specified time interval 1720. Additionally, in some embodiments, the frequency bands can be known sympathetic frequency bands associated with the design and/or construction of the arm assembly 1300. For example, in some embodiments the first specified frequency band can correspond to a sympathetic frequency of the arm assembly 1300. Similarly, the second specified frequency band can correspond to a sympathetic frequency of a base (e.g., the manipulator unit 1200) supporting the arm assembly 1300.

As depicted at 1746, in some embodiments, the filtered output signal and the filtered displacement signal can be employed by the controller 1180 to determine an adaptive gain 1748. The adaptive gain 1748 can be determined as provided by Eq. 1, wherein the filtered output signal equals the sum of an estimated bias (along a principal axes around a specified frequency band) and the product of the filtered displacement multiplied by an estimated gain (along the principal axis around the specified frequency band).

F_{y,f1}=\hat{k}_{y,f1}δy_{rc,f1}+\hat{b}_{y,f1} Eq. )1)

In an embodiment such as depicted by Eq. 1, F_{y,f1} is a known value corresponding to the filtered output signal and is indicative of a force component measured by the force sensor unit 1800 along the y-axis at the first frequency band. δy_{rc,f1} is a known value corresponding to the filtered displacement of the actual remote center RC_{A} at the first frequency band. \hat{k}_{y,f1} is an unknown value corresponding to an estimated gain on the Y component around the first frequency band. \hat{b}_{y,f1} is an unknown value corresponding to an estimated bias on the Y component around the first frequency band, which is expected to approach zero and, therefore, can be employed to monitor the safety of the gain estimator. The adaptive gain 1748 at each specified frequency and along each principal axis (e.g., adaptive gain 1748a and adaptive gain 1748b) can, for example, be determined by solving for the unknown values of Eq. 1 via a recursive least-squares with forgetting factor. It should be appreciated that the adaptive gain can be continuously computed, in real time during an operation of the system 1000. As depicted at 1750, the adaptive gain 1748 can be combined with the filtered displacement signal to determine the vibratory component 1704 of the sensed magnitude.

As depicted in FIGS. 10B and 11B, in some embodiments, the controller 1180 is configured to determine the vibratory component 1704 of the sensed magnitude of forces affecting the instrument 1400 based, at least in part, on the output signal 1702, the displacement 1714 of the portion 1401 from the nominal position (e.g., the designated position) and the second linear velocity vector 1738. For example, the controller 1180 is configured to determine the vibratory component 1704 of the sensed magnitude based on the output signal 1702, the second linear velocity vector 1738, and the displacement 1714, as determined from the second linear velocity vector 1738, of the actual remote center RCA relative to the remote center RC in the designated reference frame DF. In order to determine the vibratory component 1704, in some embodiments, the controller 1180 can be configured, via the force-modifier module 1195, to filter portions of the output signal 1702 that are indicative of the displacement and the velocity of the portion 1401 of the instrument 1400. Accordingly, as depicted at 1742 in FIGS. 10B and 11B, the controller 1180 is configured to apply a band-pass filter BP to the output signal 1702 to generate a filtered output signal. At 1744, the same band-pass filter BP is also applied to the signal indicative of the displacement 1714 of the portion 1401 of the instrument 1400 to generate a filtered displacement signal 1749. At 1745, the same band-pass filter BP is also applied to the second linear velocity vector 1738 to generate a filtered velocity signal 1751.

As depicted at 1746, in some embodiments, the filtered output signal, the filtered displacement signal, and the filtered velocity signal can be employed by the controller 1180 to determine an adaptive gain 1748. The adaptive gain 1748 can be determined as provided by Eq. 2, wherein the filtered output signal equals the sum of an estimated bias (along a principal axes around a specified frequency band) and the product of the filtered displacement multiplied by an estimated gain (along the principal axis around the specified frequency band) and the product of the filtered velocity multiplied by an estimated gain (along the principal axis around the specified frequency band).

F_{y,f1}=\hat{k}_{p1,f1}δy_{rc,f1}+\hat{k}_{v1,f1}δ\dot{y}_{rc,f1}+\hat{b}_{y,f1} Eq. (2)

In an embodiment such as depicted by Eq. 2, F_{y,f1} is a known value corresponding to the filtered output signal and is indicative of a force component measured by the force sensor unit 1800 along the y-axis at the first frequency band. δy_{rcf1} is a known value corresponding to the filtered displacement of the actual remote center RCA at the first frequency band. \hat{k}_{p1,f1} is an unknown value corresponding to an estimated gain on the Y component around the first frequency band corresponding to displacement. δ\dot{y}_{rc,f1} is a known value corresponding to the filtered velocity of the actual remote center RC_{A} at the first frequency band. \hat{K}_{v1,f1} is an unknown value corresponding to an estimated gain on the Y component around the first frequency band corresponding to velocity. \hat{b}_{y,f1} is an unknown value corresponding to an estimated bias on the Y component around the first frequency band, which is expected to approach zero and, therefore, can be employed to monitor the safety of the gain estimator. The adaptive gain 1748 at each specified frequency and along each principal axis can, for example, be determined by solving for the unknown values of Eq. 2 via a recursive least-squares with forgetting factor. It should be appreciated that the adaptive gain can be continuously computed, in real time during an operation of the system 1000. As depicted at 1750, the adaptive gain 1748 can be combined with the filtered displacement signal 1749 and the filtered velocity signal 1751 to determine the vibratory component 1704 of the sensed magnitude.

As depicted in FIG. 10C, in some embodiments, the controller 1180 is configured to determine the vibratory component 1704 of the sensed magnitude of forces affecting the instrument 1400 based, at least in part, on the output signal 1702 and the second linear velocity vector 1738. For example, the controller 1180 is configured to determine the vibratory component 1704 of the sensed magnitude based on the output signal 1702 and the second linear velocity vector 1738 of the remote center RC in the designated reference frame DF. In order to determine the vibratory component 1704, in some embodiments, the controller 1180 can be configured, via the force-modifier module 1195, to filter portions of the output signal 1702 that are indicative of the velocity of the portion 1401 of the instrument 1400. Accordingly, as depicted at 1742 in FIG. 10C, the controller 1180 is configured to apply a band-pass filter BP to the output signal 1702 to generate a filtered output signal. At 1745, the same band-pass filter BP is also applied to the second linear velocity vector 1738 to generate a filtered velocity signal.

As depicted at 1746, in some embodiments, the filtered output signal and the filtered velocity signal can be employed by the controller 1180 to determine an adaptive gain. The adaptive gain can be determined as provided by Eq. 3, wherein the filtered output signal equals the sum of an estimated bias (along a principal axis around a specified frequency band) and the product of the filtered velocity multiplied by an estimated gain (along the principal axis around the specified frequency band).

F_{y,f1}=\hat{k}_{v1,f1}δ\dot{y}_{rc,f1}+\hat{b}_{y,f1} Eq. (3)

In an embodiment such as depicted by Eq. 3, F_{y,f1} is a known value corresponding to the filtered output signal and is indicative of a force component measured by the force sensor unit 1800 along the y-axis at the first frequency band. δy_{rc,f1} is a known value corresponding to the filtered velocity of the actual remote center RC_{A} at the first frequency band. \hat{k}_{v1,f1} is an unknown value corresponding to an estimated gain on the Y component around the first frequency band corresponding to velocity. \hat{b}_{y,f1} is an unknown value corresponding to an estimated bias on the Y component around the first frequency band, which is expected to approach zero and, therefore, can be employed to monitor the safety of the gain estimator. The adaptive gain 1748 at each specified frequency and along each principal axis can, for example, be determined by solving for the unknown values of Eq. 3 via a recursive least-squares with forgetting factor. It should be appreciated that the adaptive gain can be continuously computed, in real time during an operation of the system 1000. As depicted at 1750, the adaptive gain 1748 can be combined with the filtered displacement signal 1749 and the filtered velocity signal 1751 to determine the vibratory component 1704 of the sensed magnitude. As depicted at 1752, in some embodiments, the plurality of operations can include determining a gain state of the haptic feedback module 1196. The gain state of the haptic feedback module can determine the degree to which the magnitude of the force delivered to the operator of the system 1000 coincides with the magnitude of the forces affecting the instrument 1400. For example, at a low-gain state the haptic feedback will represent a relatively low percentage (e.g., less than 40%) of the force magnitude as sensed by the force sensor unit 1800. When the system 1000 is operated in a mid-gain state, the haptic feedback may represent between 40 and 60% of the force affecting the instrument 1400. Additionally, at a high-gain state, the haptic feedback can represent greater than 60% of the force magnitude affecting the instrument 1400. In the high-gain state, fluctuations in the force affecting the instrument 1400, such as may manifest from the displacement of the portion 1401 of the instrument 1400, can be presented to the operator, while in the low-gain state such fluctuations may be damped by the haptic feedback module 1196. Accordingly, at 1754, a tuning gain 1756 can be determined based, at least in part, on the gain state of the haptic feedback module 1196. As depicted at 1758, the tuning gain 1756 can be applied to the vibratory component 1704 in order to establish the portion of the vibratory component 1704 that is filtered out at 1708. In other words, the tuning gain 1756 can be used to coordinate the degree to which the vibratory component is filtered with the gain state of the system 1000. For example, when the gain state of the haptic feedback module 1196 is a high-gain state, the tuning gain 1756 can be 0.8 or greater to maximize the filtering of the vibratory component 1704 thereby minimizing the portion of the sensed magnitude resulting from the displacement of the instrument 1400 that is used to determine the magnitude of the haptic feedback. However, when the gain state of the haptic feedback module 1196 is a low-gain state, the inherent damping of the output signal 1702 can reduce the desirability of further filtering the vibratory component 1704.

FIG. 13 is a flow chart of a method 60 of control for a surgical system according to an embodiment. The method 60 may, in an embodiment, be performed via a teleoperated system, such as system 1000 as described with reference to FIGS. 1-12. However, it should be appreciated that in various embodiments, aspects of the method 60 may be accomplished via additional embodiments of the system 1000 or components thereof as described herein. Accordingly, the method 60 may be implemented on any suitable device as described herein. Thus, the method 60 is described below with reference to instrument 1400 and the controller 1180 of the system 1000 as previously described, but it should be understood that the method 60 can be employed using any of the medical devices/instruments and controllers described herein.

As depicted at 61, the method 60 includes determining, via the controller, a coordinate location for a nominal position of a portion of the medical instrument. The coordinate location for the nominal position remains unchanged throughout a procedure. As depicted at 62, the method 60 includes receiving, via the controller, a linear acceleration vector from a motion tracker configured to monitor a motion of the portion of the medical instrument. As depicted at 63, the method 60 includes receiving, via the controller, an angular velocity vector from the motion tracker. As depicted at 64, the method 60 includes determining, via a displacement module of the controller, a displacement of the portion of the medical instrument relative to the nominal position based on the linear acceleration vector and the angular velocity vector. As depicted at 65, the method 60 includes modifying, via the controller, an operation of the surgical system based on the displacement of the portion of the medical instrument from the nominal position. For example, modifying the operation of the surgical system can include at least one of modifying a haptic feedback provided to the input device, initiating a calibration sequence, applying a damping factor to a commanded movement of the surgical system, halting an operation of the surgical system, generating an error signal, and generating a service request for the surgical system.

As shown particularly in FIG. 12, a schematic diagram of one embodiment of suitable components that may be included within the controller 1180 is illustrated. In some embodiments, the controller 1180 is positioned within a component of the surgical system 1000, such as the user control unit 1100 and/or the optional auxiliary equipment unit 1150. However, the controller 1180 may also include distributed computing systems wherein at least one aspect of the controller 1180 is at a location which differs from the remaining components of the surgical system 1000 for example, at least a portion of the controller 1180 may be an online controller.

As depicted, the controller 1180 includes one or more processor(s) 1182 and associated memory device(s) 1184 configured to perform a variety of computer implemented functions (e.g., performing the methods, steps, calculations and the like and storing relevant data as disclosed herein). Additionally, in some embodiments, the controller 1180 includes a communication module 1186 to facilitate communications between the controller 1180 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1184 may generally comprise memory element(s) including, but not limited to, computer readable medium (e.g., random access memory (RAM)), computer readable nonvolatile medium (e.g., a flash memory), a floppy disc, a compact disc read only memory (CD ROM), a magneto optical disc (MOD), a digital versatile disc (DVD) and/or other suitable memory elements. Such memory device(s) 1184 may generally be configured to store suitable computer readable instructions that, when implemented by the processor(s) 1182, configure the controller 1180 to perform various functions.

In some embodiments, the controller 1180 includes a haptic feedback module 1820. The haptic feedback module 1820 may be configured to deliver a haptic feedback to the operator based on inputs received from a force sensor unit 1800 of the instrument 1400. In some embodiments, haptic feedback module 1820 may be an independent module of the controller 1180. However, in some embodiments the haptic feedback module 1820 may be included within the memory device(s) 1184.

The communication module 1186 may include a control input module 1188 configured to receive control inputs from the operator/surgeon S, such as via the input device 1116 of the user control unit 1100. The communication module may also include an indicator module 1192 configured to generate various indications in order to alert the operator.

The communication module 1186 may also include a sensor interface 1190 (e.g., one or more analog to digital converters) to permit signals transmitted from one or more sensors (e.g., strain sensors of the force sensor unit 1800) to be converted into signals that can be understood and processed by the processors 1182. The sensors may be communicatively coupled to the communication module 1186 using any suitable means. For example, the sensors may be coupled to the communication module 1186 via a wired connection and/or via a wireless connection, such as by using any suitable wireless communications protocol known in the art. Additionally, in some embodiments, the communication module 1186 includes a device control module 1194 configured to modify an operating state of the instrument 1400 (and/or any of the instruments described herein. Accordingly, the communication module is communicatively coupled to the manipulator unit 1200 and/or the instrument 1400. For example, the communications module 1186 may communicate to the manipulator unit 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a set of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments, however, any of the links, tool members, beams, shafts, cables, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A surgical system, comprising:
a medical instrument supported by a manipulator unit;
a force sensor unit coupled to a distal end portion of the medical instrument;
an input device operably coupled to the medical instrument; and
a controller operably coupled to the manipulator unit, the input device, and the force sensor unit;
wherein the controller comprises a force-modifier module and a haptic feedback module; and
wherein the controller performs a plurality of operations comprising:
receiving an output signal from the force sensor unit, the output signal corresponding to a sensed magnitude of forces exerted on the medical instrument, the output signal including a vibratory component of the sensed magnitude and a commanded-motion component of the sensed magnitude,
determining, via the force-modifier module, the vibratory component corresponding to a non-commanded motion of the medical instrument,
generating, via the force-modifier module, a modified force-magnitude signal by filtering out the vibratory component, the modified force-magnitude signal corresponding to a force resulting from a commanded motion of the medical instrument, and
providing, via the haptic feedback module, a haptic feedback to the input device based on the modified force-magnitude signal.

2. The system of claim 1, wherein:
the system further comprises a motion tracker operably coupled to the controller;
the motion tracker is configured to measure a displacement of a portion of the medical instrument as expressed in a designated reference frame; and
the vibratory component of the sensed magnitude is correlated with the displacement of the portion of the medical instrument during a time interval.

3. The system of claim 2, wherein:
the manipulator unit includes an arm assembly;
the arm assembly includes a spar supporting the medical instrument;
the motion tracker includes an inertial measurement unit coupled to the spar; and
the displacement of the portion of the medical instrument is associated with both an acceleration output and an angular rate output of the inertial measurement unit.

4. The system of claim 1, wherein:
the non-commanded motion of the medical instrument is associated with a displacement of a portion of the medical instrument from a nominal position;
the displacement during a time interval corresponds to a vibratory displacement of the portion of the medical instrument; and
the vibratory component of the sensed magnitude is determined based at least in part on the vibratory displacement of the portion of the medical instrument.

5. (canceled)

6. The system of claim 4, wherein:
the system includes a motion tracker operably coupled to the controller;
the controller includes a displacement module;
the plurality of operations includes receiving, via the displacement module, one or more inputs from the motion tracker;
the one or more inputs are associated with the displacement of the portion of the medical instrument from the nominal position as expressed in a designated reference frame; and
the one or more inputs include an indication of a linear acceleration vector and an indication of an angular velocity vector of the portion of the medical instrument.

7. (canceled)

8. The system of claim 6, wherein:
the manipulator unit includes an arm assembly;
the arm assembly includes a spar supporting the medical instrument;
the motion tracker is coupled to the spar at a mounting position;
the mounting position is separated from the portion of the medical instrument; and
the plurality of operations further comprises:
determining a first linear velocity vector of the motion tracker at the mounting position based on the linear acceleration vector from the motion tracker,
generating a modified angular velocity vector by applying a position constant to the angular velocity vector of the motion tracker, the position constant correlating the angular velocity vector of the motion tracker to an angular velocity of the portion of the medical instrument,
combining the first linear velocity vector with the modified angular velocity vector to determine a second linear velocity vector of the portion of the medical instrument, and
determining the displacement of the portion of the medical instrument from the nominal position based on the second linear velocity vector.

9. The system of claim 4, wherein the plurality of operations includes:
determining, via the force-modifier module, the vibratory component of the sensed magnitude based at least in part on the output signal and the displacement of the portion of the medical instrument from the nominal position.

10. The system of claim 9, wherein the plurality of operations includes:
applying, via the force-modifier module, a band-pass filter to the output signal to generate a filtered output signal;
applying, via the force-modifier module, the band-pass filter to a signal indicative of the displacement of the portion of the medical instrument to generate a filtered displacement signal;
determining, via the force-modifier module, an adaptive gain based at least in part on the filtered output signal and the filtered displacement signal; and
combining the adaptive gain with the filtered displacement signal to determine the vibratory component of the sensed magnitude.

11-13. (canceled)

14. The system of claim 1, wherein:
the plurality of operations includes determining a gain state of the haptic feedback module;
the filtering out of the vibratory component of the sensed magnitude includes applying a tuning gain to the vibratory component; and
the tuning gain is based at least in part on the gain state of the haptic feedback module.

15. (canceled)

16. The system of claim 1, wherein:
the system further comprises a motion tracker operably coupled to the controller;
the motion tracker is configured to measure a velocity of a portion of the medical instrument as expressed in a designated reference frame; and
the vibratory component of the sensed magnitude is correlated with the velocity of the portion of the medical instrument.

17. A method of control for a surgical system, the surgical system including a manipulator unit, a controller, an input device, and a medical instrument, the manipulator unit including an arm assembly supporting the medical instrument, and the medical instrument being operably coupled to be controlled by the input device via the controller, the method comprising:
determining, via the controller, a coordinate location for a nominal position of a portion of the medical instrument, the coordinate location for the nominal position remaining unchanged throughout a medical procedure;
receiving from a motion tracker, via the controller, a linear acceleration vector;
receiving from the motion tracker, via the controller, an angular velocity vector;
determining, via a displacement module of the controller, a displacement of the portion of the medical instrument relative to the nominal position based on the linear acceleration vector and the angular velocity vector; and
modifying, via the controller, an operation of the surgical system based on the displacement of the portion of the medical instrument from the nominal position.

18. The method of claim 17, wherein:
determining the displacement relative to the nominal position includes determining a displacement magnitude along three axes; and
the three axes are orthogonal to one another.

19-20. (canceled)

21. The method of claim 17, wherein:
the arm assembly includes a spar supporting the medical instrument;
the motion tracker is coupled to the spar at a mounting position;
the mounting position is separated from the portion of the medical instrument; and
the determining of the displacement of the portion of the medical instrument includes:
determining a first linear velocity vector of the motion tracker at the mounting position based on the linear acceleration vector,
generating a modified angular velocity vector by applying a position constant to the angular velocity vector, the position constant correlating the angular velocity vector of the motion tracker to an angular velocity of the portion of the medical instrument,
determining a second linear velocity vector of the portion of the medical instrument by combining the first linear velocity vector with the modified angular velocity vector, and
determining the displacement of the portion of the medical instrument from the nominal position based on the second linear velocity vector of the portion of the medical instrument.

22. The method of claim 17, wherein:
modifying the operation of the surgical system includes at least one of modifying a haptic feedback provided to the input device, initiating a calibration sequence, applying a damping factor to a commanded movement of the surgical system, halting an operation of the surgical system, generating an error signal, and generating a service request for the surgical system.

23. A method of control for a surgical system, the surgical system including a manipulator unit, a controller, an input device, and a medical instrument, the manipulator unit including an arm assembly supporting the medical instrument, the medical instrument being operably coupled to be controlled by the input device via the controller, and the medical instrument including a force sensor unit, the method comprising:
receiving, via the controller, an output signal from the force sensor unit, the output signal corresponding to a sensed magnitude of forces exerted on the medical instrument, the output signal including a vibratory component of the sensed magnitude and a commanded-motion component of the sensed magnitude,
determining, via a force-modifier module of the controller, the vibratory component corresponding to a non-commanded motion of the medical instrument;
generating, via a force-modifier module of the controller, a modified force-magnitude signal by filtering out the vibratory component, the modified force-magnitude signal corresponding to a force resulting from a commanded motion of the medical instrument; and
providing, via a haptic feedback module of the controller, a haptic feedback to the input device based on the modified force-magnitude signal.

24-25. (canceled)

26. The method of claim 23, wherein:
the non-commanded motion of the medical instrument is associated with a displacement of a portion of the medical instrument from a nominal position;
the displacement during a time interval corresponds to a vibratory displacement of the portion of the medical instrument; and
the vibratory component of the sensed magnitude is determined based at least in part on the vibratory displacement of the portion of the medical instrument.

27-30. (canceled)

31. The method of claim 26, wherein:
the method includes determining, via the force-modifier module, the vibratory component of the sensed magnitude based at least in part on the output signal and the displacement of the portion of the medical instrument from the nominal position.

32. The method of claim 31, wherein:
the method includes:
applying, via the force-modifier module, a band-pass filter to the output signal to generate a filtered output signal;
applying, via the force-modifier module, the band-pass filter to a signal indicative of the displacement of the portion of the medical instrument to generate a filtered displacement signal;
determining, via the force-modifier module, an adaptive gain based at least in part on the filtered output signal and the filtered displacement signal; and
combining the adaptive gain with the filtered displacement signal to determine the vibratory component of the sensed magnitude.

33. The method of claim 32, wherein:
the band-pass filter is one of a plurality of band-pass filters;
each band-pass filter of the plurality of band-pass filters is associated with a specified frequency band of a plurality of specified frequency bands;
each specified frequency band of the plurality of specified frequency bands is associated with a known vibratory displacement of the medical instrument; and
the force-modifier module determines the vibratory component at each of the plurality of specified frequency bands.

34. The method of claim 33, wherein:
the non-commanded motion of the medical instrument results from a compliance of the manipulator unit.

35-39. (canceled)
